(19) United States
(12) Patent Application Publication
(10) Pub. No.: US 2021/0034841 A1
(43) Pub. Date: Feb. 4, 2021

(54) SYSTEM AND METHOD FOR AUTOMATED PREDICTION OF DIFFICULT AIRWAY MANAGEMENT USING IMAGES

(71) Applicant: Wake Forest University Health Sciences, Winston-Salem, NC (US)

(72) Inventors: B. Scott Segal, Winston-Salem, NC (US); Muhammad Khalid Khan Niazi, Winston-Salem, NC (US)

(21) Appl. No.: 16/944,789

(22) Filed: Jul. 31, 2020

Related U.S. Application Data
(60) Provisional application No. 62/881,065, filed on Jul. 31, 2019.

(51) Int. Cl. G06K 9/00; A61B 5/00; G06N 3/08

(52) U.S. Cl. CPC G06K 9/00275; A61B 5/48; G06N 3/08; G06K 9/00288; A61B 5/7275

(57) ABSTRACT

Disclosed are systems and methods for automated analysis of facial photographs for improved airway management and patient safety using unsupervised computer algorithms based on feature extraction from facial photographs by deep-learning algorithms. A deep-learning algorithm-based feature extractor uses frontal and/or profile views of the face to identify important information about potential intubation difficulty. This information is used by a trained advanced algorithm to classify faces as easy or difficult to intubate based on the extracted features.

### CROSS-REFERENCE TO RELATED APPLICATION

This application claims priority to and benefit of U.S. provisional patent application Ser. No. 62/881,065 filed Jul. 31, 2019, which is fully incorporated by reference and made a part hereof.

### BACKGROUND

Successful airway management is fundamental to safe anesthetic performance, and airway management failure continues to be the leading cause of anesthesia-related death and severe morbidity. Many perioperative airway mishaps in the emergency department, ICU, and patient floor also contribute to morbidity and mortality. While preoperative airway assessment is considered the worldwide standard of care, 75-93% of difficult intubations are unanticipated. Unfortunately, all easily performed airway examination systems in clinical practice perform only modestly, with sensitivities of 20-62%, specificities of 82-97%, and very low positive predictive values, generally less than 30%, unless very liberal definitions of difficulty are used. Even multivariable combinations of these tests fail to improve predictive performance. There are likely a number of reasons for this poor performance, including the relative rarity of difficult intubation, the multifactorial etiology and varying definition of difficult intubation, inter-observer variability in test results, failure to validate potential systems in patients independent of those used to derive the test, and the inadequacy of the tests themselves.

Predicting difficult airway management (endotracheal intubation) is considered both essential to safe practice and a standard of care, but present bedside tests are inadequate in terms of sensitivity and positive predictive value. Endotracheal intubation occurs millions of times a year, in both elective and emergency situations, in the operating room, ICU, emergency department, and paramedic squads. Approximately 5-10% of intubations are difficult, depending on the definition, and accurate prediction would allow the clinician to secure advanced airway equipment or specially trained personnel to assist, preventing a potential airway disaster. Unfortunately, as noted above, 75-93% of difficult intubations are unanticipated, so an accurate method for prediction would be a considerable advance.

Therefore, systems and methods are desired that overcome challenges in the art, some of which are described above. There is a need to improve on existing methods of airway evaluation by utilizing the power of machine learning to extract features of facial anatomy from subject photographs, which in some instances may be combined with bedside examination results, to construct a superior airway prediction algorithm. Because airway mishaps are both the most common, and likely most preventable, causes of anesthesia-related morbidity and mortality, such an innovation could have marked benefits to patients. In addition, the avoidance of unnecessary awake intubation, use of advanced airway equipment, or other painful or expensive maneuvers could reduce patient discomfort and healthcare system cost.

### SUMMARY

Successful airway management is fundamental to safe anesthetic performance, and airway management failure continues to be the leading cause of anesthesia-related death and severe morbidity. While preoperative airway assessment is considered the worldwide standard of care, 75-93% of difficult intubations are unanticipated, and all easily performed airway examination systems in clinical practice perform only modestly.

Disclosed and described herein are embodiments of a machine learning system based on analysis of facial photographs for improved airway management and patient safety. Previous work has demonstrated that an algorithm based on supervised (i.e., human assisted) computer analysis of facial images combined with thyromental distance (TMD) can outperform classical bedside tests and human experts. Here, this work has been extended by development of completely unsupervised computer algorithms based on feature extraction from facial photographs by deep-learning algorithms (e.g., convoluted neural networks (CNNs) and/or convolutional autoencoders (CAE)). CNN technology already exists for highly accurate deterministic feature extraction of frontal views of the face and is widely employed in facial recognition applications. A similar CNN-based feature extractor is used herein from profile views of the face, which contains important information about potential intubation difficulty. This information is fused with frontal facial information and patient demographics and bedside airway data (TMD and Mallampati class (MP)) and used to train an advanced algorithm to classify faces as easy or difficult to intubate based on prospective observation of ground truth during induction of general anesthesia.

One implementation described herein comprises a smartphone-based data entry tool to capture photographs, patient demographic information, and bedside airway examination data and transmit it to an online database. This forms the basis of a completely automated airway prediction tool, based on our methods described herein.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a flowchart that illustrates and exemplary method of predicting the difficulty of airway management from frontal and/or profile views of facial images;

FIG. 2 shows 14 out of the 47 anthropometric facial landmarks on frontal and profile view images;

FIG. 3 is an illustration of the general framework of a convolutional encoder; and

FIG. 4 illustrates an exemplary computer that can be used for performing the computational steps described herein.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

As used herein, “anesthesiologist” or “anesthesiology assistant” refers to any medical professional skilled in airway assessment and management, regardless of education, training, licensure, rank or title.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

Prior work has demonstrated that analysis of facial structure by a supervised computer program can generate an algorithm capable of markedly outperforming bedside tests (MP+TMD) as well as human experts given the same information. For example, photographs of 80 subjects' faces, 40 easy and 40 difficult, were analyzed by software (FaceGen Modeler v3.3; Singular Inversions, Vancouver) that resolves each face into 61 facial proportions derived from an algorithm that models the face as a single point in a 50-dimensional eigenspace. A difficult airway was defined liberally, which increases the difficulty of discrimination, as more than 1 attempt by an experienced operator with a Macintosh 3 laryngoscope, grade 3 or 4 laryngoscopic view, or non-elective use of an adjunctive airway device. Each parameter was tested individually for discriminatory ability by logistic regression, and combinations of 11 variables with P<0.1, plus MP class and TMD, were tested exhaustively by all possible binomial quadratic logistic regression models. Candidate models were cross-validated by maximizing the product of the area under the receiver operating characteristic curves obtained in the derivation and validation cohorts. AUC of the ROC curve was used rather than raw accuracy or sensitivity alone to maximize accuracy of future predictions. The best model contained just 3 facial parameters and TMD, and correctly classified 70 of 80 subjects (P<10⁻⁸). In contrast, the combination of MP and TMD correctly classified 47 of 80 (P=0.073). Sensitivity, specificity, and area under the curve for the computer model were 90%, 85%, and 0.899, respectively. Inthe subset of more difficult airways (Intubation Difficulty Scale [IDS]²²>5), the model demonstrated values of 96%, 85%, and 0.902.

Described herein is the use of an unsupervised deep-learning algorithm) e.g., CNN, CAE, etc.) based feature extraction rather than human-supervised computerized image analysis. In one example, subjects were reanalyzed with ground truth who were studied in the aforementioned investigations. The frontal views of each face were first pre-processed by Python algorithms which drew a bounding box around the face, reduced the resolution to 256×256 pixels, and reduced the color depth to 256 shades of gray. Then each preprocessed image was analyzed by the OpenFace CNN, which returned a 128-element matrix of signed real numbers deterministically characterizing the face. An analysis platform (RapidMiner Studio, version 8.0, Boston, Mass.) was used to construct a predictive algorithm for easy vs. difficult airways from the frontal face parameters, bedside airway exams (MP, TMD), and patient demographics (age, height, weight, BMI). Evolutionary feature selection was used (tournament scheme, tournament size 0.25) restricting the selection to 20 attributes. Naïve Bayes models were selected with 20-fold cross validation. Bonferroni correction for multiple models (N=226) was applied. A model including 18 OpenFace parameters, age, and TMD correctly classified 65/80 faces (corrected P=3.5×10⁻⁷). Sensitivity, specificity, and AUC were 87.5%, 75%, and 0.8. This analysis was sharply limited by excluding all profile view information about the face, which may be very important in airway prediction, because it contains potentially relevant information on, for example, jaw length and face and neck angles.

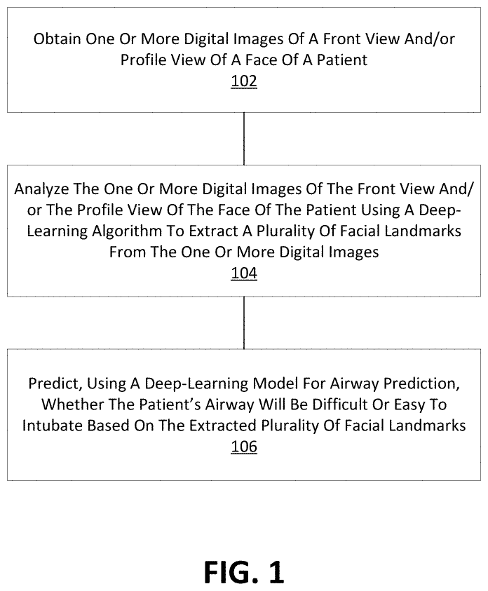

FIG. 1 is a flowchart that illustrates an exemplary method of predicting the difficulty of airway management from frontal and/or profile views of facial images, as described herein. At step 102, one or more digital images of a candidate patient are obtained. These images may be obtained using a camera or any other device capable of obtaining digital images of the face of a person. The images may comprise frontal and/or profile images of the candidates face. At 104, the images are analyzed by trained deep-learning algorithms to extract facial landmarks from the one or more digital images. For example, the deep-learning algorithms may comprise a computer executing convolutional autoencoder (CAE) software. The CAE software extracts facial landmarks from the one or more digital images. In some instances, the facial landmarks comprise Farkas's anthropometric facial landmarks from frontal and/or profile images.

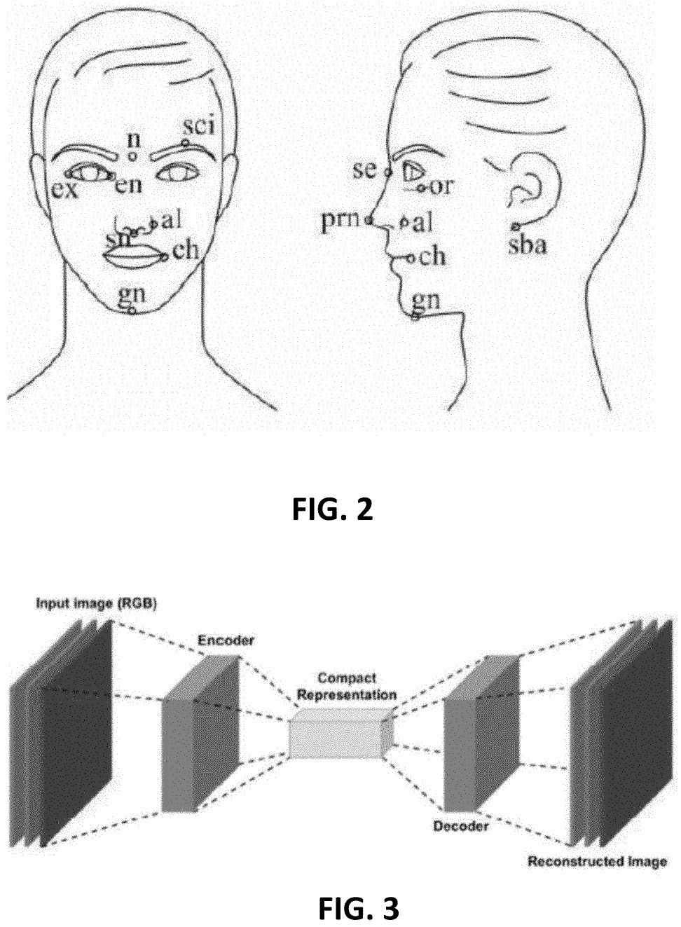

Farkas's anthropometric facial landmarks are often manually identified to quantify face morphology through anthropometric measurements and have been shown to be clinically significant in identifying anatomical differences among different subpopulations which affect choices during aesthetic maxillofacial surgery. Thus, these landmarks can be used in identifying patients who are at high risk of difficult intubation. The Farkas's system uses a total of 47 landmark points to describe a face. The landmarks are typically identified by abbreviations of corresponding anatomical terms. For example, the inner corner of the eye is abbreviated as “en” for endocanthion, while the outer corner is “ex” for exocanthion, the most anterior point of the frontonasal suture that joins the nasal part of the frontal bone and the nasal bones is denoted as “n” (nasion). Similarly, the landmark that describes the midpoint of the lower border of the human mandible is known as “gn” (gnathion) while the landmark where the nasal septum and the upper lip meet in the midsagittal plane is “sn” subnasa/e. The landmark located directly above the most lateral point of the iris is denoted as “sci” (uperciliare) while a/(a/are) represents the lateral point on the flare of the nose. FIG. 2 shows 14 anthropometric facial landmarks on frontal and profile view images. Moreover, in some instances, extracted landmarks may include identifying mantum, hyoid bone, and thyroid notch to encode the neck area. It is a common practice in anesthesiology to use these landmarks for anthropometric measurements to identify difficult to intubate patients. However, unlike anesthesiologists, these landmarks are used to synthetically generate a facial image that will be used for computing the risk of difficult intubation. As noted above, the landmarks are identified with an ensemble of deep convolutional autoencoders (CAEs). In some instances, FN landmarks will refer to 50 landmarks (Farkas's anthropometric facial landmarks and neck landmarks).

An autoencoder is an unsupervised deep-learning algorithm which relies on back propagation to learn a function that maps an input to itself, i.e. an approximation of the identify function. It might seem trivial to learn such a function, but by placing constraints on the neurons (within layers), interesting patterns within images can be automatically learned. The constraints can be in the form of compact representation or in terms of sparsity (constraining the neurons to be inactive most of the time). Structure-wise, an autoencoder is a fully-connected feed-forward neural network comprised of an input layer, a hidden layer(s), and an output layer (the same size as the input). FIG. 3 is an illustration of the general framework of a convolutional encoder. The encoder maps the input to the hidden layer, and the decoder maps the encoded hidden layer to the output. The idea is that this output should represent a reconstruction of the original input image. The difference between this reconstructed output and the original input constitutes what is known as a loss function. The mapping functions originally applied in the decoder and encoder are then tweaked in order to minimize this loss, thus driving the training and optimization process. Given that the input and output images are the same, this training process is unsupervised. Often, the number of hidden layer units is smaller than the size of the input and output images. This forces the autoencoder to learn a latent (i.e. compact) representation of the input (see FIG. 3). It is in this hidden layer bottleneck that a set of basis functions (both encoding and decoding) for the training dataset are learned.

CAEs combine the ideas of unsupervised reconstruction of input images and bottlenecking with convolution. Specifically, rather than learning a set of basis functions for images, a set of convolutional filters are learned in a manner similar to the convolutional filters learned by convolutional neural networks (CNNs). As an image is presented to a CAE, a set of convolutional filters are applied, followed by activation and pooling. As in CNNs, these steps are repeated some number of times, forming the encoder. This is followed by some number of fully connected layers, forming a bottleneck. However unlike CNNs, rather than performing classification on the outputs of the final fully connected layer, a series of deconvolution filters, activations, and up-samplings are performed, opposite of the convolutional steps, forming the decoder. This results in an output the same size as the input image. And as in autoencoders, a cost function measures the difference between the input image and this output, driving the learning of filters. In this manner, the training of convolutional autoencoders is unsupervised. Once a CAE is trained using this unsupervised method, it may be used as a feature extractor for subsequent training of a classifier. The feature extraction step can be carried out by simply passing a set of labeled images through the CAE and keeping the outputs of the fully connected layers, ignoring the decoder. These labeled feature vectors may then be used by any number of classifiers.

At 104, FN landmarks are identified from the one or more frontal and/or profile images of faces using deep-learning algorithms such as CAEs and CNNs. For training the CAEs, an experienced anesthesiology assistant annotates these landmarks on facial images. One CAE is trained per FN landmark. In other words, if there are 50 FN landmarks, then there are 50 trained CAEs corresponding to 50 FN landmarks. Facial images are divided into patches using stereology to extract FN landmarks for training of CAEs. Following training, the (trained) CAEs as used as feature extractors. Unlike conventional CAE, this methodology ensures that each CAE learns a set of features that are specific to a certain FN landmark. Finally, a softmax layer (after the addition of a few fully connected layers) is trained to classify a given feature vector into a fiducial landmark class. This proposed method is markedly different from conventional methods in that a CAE is trained per class only on that class of patches.

The classifier described above is used to detect FN landmarks in frontal and profile images of faces. This is carried out by passing a window (the size of the CAE input) over an input image and applying the classifier to this windowed image. For each window, the softmax layer outputs a per-class probability. The highest probability in the softmax output corresponds to the class of the windowed image. In order to classify a particular windowed image as background (i.e. not part of any FN landmarks), an empirically-determined threshold for the softmax output is applied. As each component of the softmax output vector reflects a per class probability for the identity of a particular image, when no probability is particularly high (i.e. below the threshold), it is labeled as background class. Through these methods, facial landmarks are localized.

Inspired by random forest, the above methods are applied to build an ensemble of classifiers and detectors. The original dataset of frontal and profile facial images is randomly sampled with replacement to create several independent datasets. Each dataset is then individually subjected to the methods described above (CAE training, CAE feature extraction, additional of a few fully connected layers, training of softmax classifier), creating an ensemble of classifiers and detectors. As a window is presented to the ensemble, each classifier makes a prediction, and the final classification is determined by majority voting among these ensemble classifiers.

Returning to FIG. 1, at step 106 the identified FN landmarks are used to learn the underlying distribution of both easy and difficult to intubate patients. This is accomplished in some instances through generative adversarial networks (GANs). GANs are a class of unsupervised machine learning algorithms in which two neural networks are pitted against one another in a zero sum (i.e. adversarial) game. These two networks are referred to as the generator (G) and the discriminator (D). A discriminator is a familiar concept—it learns to map features to class labels. For example, modern CNNs attempt to classify images into specific categories. Generators are the opposite—they map labels to features, generating images from categories. In GANs, the generator learns to map a latent space to the distribution of the given dataset, while the discriminator learns to differentiate between samples from the given dataset and fraudulent samples produced by the generator. The common analogy is that of a counterfeiter and an expert. The generator learns to produce better counterfeits of the dataset as the discriminator learns to spot real and counterfeited data. As both G and D are being trained, the weights in D are adjusted to maximize the probability of differentiating between real and fake images by the function log D(x), where x is the ‘real’ probability. As it is a zero-sum game, the weights in G are adjusted to minimize the negation of this value log(1−D(G(z)), where z is random noise. These two equations constitute the cross-entropy loss function. Specifically, D and G participate in a two-player minimax game described by the function:

$${min}_{G} {max}_{D} {E}_{x \sim {p}_{data} ( x )} [ log D ( x ) ] + {E}_{z \sim {p}_{z} ( z )} [ 1 - log D ( G ( z ) ] .$$

Conditional generative adversarial networks (cGANs) are used herein to determine, using the identified facial features of step 104, if the patient will be easy or difficult to intubate. cGANs are an extension of GANs in which input data for G and D are both conditioned on some additional information, in the instant case, the edge map of FN landmarks (edges extracted from the FN landmarks through Canny edge detector) serves as the condition. This is carried out by feeding this additional information into both G and D in a joint representation. Thus, the minimax function becomes:

$${min}_{G} {max}_{D} {E}_{x \sim {p}_{data} ( x )} [ log D ( x \vert y ) ] + {E}_{z \sim {p}_{z} ( z )} [ 1 - log D ( G ( z \vert y ) ]$$

where y is the additional information. The cost function is modified by addition the structural similarity index (SSIM) to ensure that the reconstructed image is a close approximation of the input image. The disclosed method conditions the input data on channel-wise Canny-detected edges. By only providing the edge (binary images) information, the cGANs are forced to reconstruct the facial images from FN landmarks. The edge condition is a 3-channel binary mask in which white pixels correspond to edges within FN landmarks. The edge condition ensures that different anatomical structures around FN are constructed at the same location as in the input image. This is desired as it enables the pixel-to-pixel comparison between the reconstructed and the input image. SSIM is used to test the anatomical correctness of the reconstructed facial image.

Two different cGANs are trained. The first cGANs is trained to reconstruct faces of patients who were easy to intubate from their corresponding FN landmarks (the Canny edge map will serve as condition). This network is trained on patient faces that were easy to intubate. Similarly, the second cGAN is trained to reconstruct faces of patients that were difficult to intubate. This network is trained on faces of patients who were difficult to intubate. Once again, the Canny edge map extracted from FN points serves as the condition for the second cGAN. Essentially, these networks are only good at reconstructing faces for patients within the same class (easy or difficult intubation). The networks will be trained on facial images and conditioned by Canny edge map resulting from FN landmarks.

In one aspect, a smartphone-based data entry tool is used to capture images, demographic information, airway examinations, and intubation results. Two approaches will be considered simultaneously. In one embodiment, an iOS-based tool is used with the source code updated to current iOS versions and persistent data storage methods. Second, a FileMaker mobile implementation is used, and the data transmitted directly to an online database. Such tools, which have the advantage of being platform agnostic.

In one aspect, the networks and the prediction engine can be implemented in a cloud computing architecture so that users could access the predictions (and/or train the network) via a mobile application which uploads photographs and demographic information and returns the prediction or helps train the network.

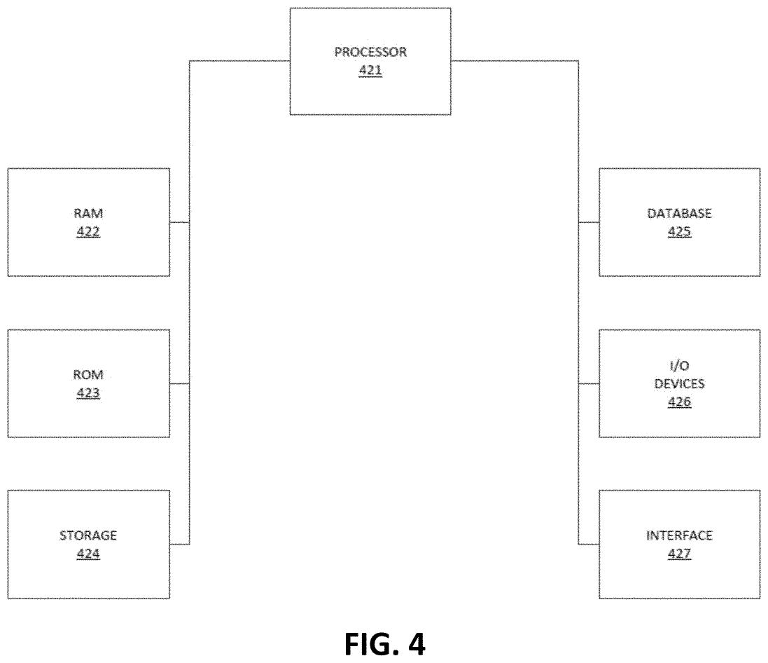

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for discriminating tissue of a specimen. In one exemplary aspect, the units can comprise a computing device that comprises a processor 421 as illustrated in FIG. 4 and described below. In some aspects, the computer of FIG. 4 may comprise all or a portion of a smartphone.

FIG. 4 illustrates an exemplary computer that can be used for making automated prediction of difficult airway management using images. As used herein, “computer” may include a plurality of computers. The computers may include one or more hardware components such as, for example, a processor 421, a random-access memory (RAM) module 422, a read-only memory (ROM) module 423, a storage 424, a database 425, one or more input/output (I/O) devices 426, and an interface 427. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 424 may include a software partition associated with one or more other hardware components. It is understood that the components listed above are exemplary only and not intended to be limiting.

Processor 421 may include one or more processors, each configured to execute instructions and process data to perform one or more functions associated with a computer for discriminating tissue of a specimen. Processor 421 may be communicatively coupled to RAM 422, ROM 423, storage 424, database 425, I/O devices 426, and interface 427. Processor 421 may be configured to execute sequences of computer program instructions to perform various processes. The computer program instructions may be loaded into RAM 422 for execution by processor 421.

RAM 422 and ROM 423 may each include one or more devices for storing information associated with operation of processor 421. For example, ROM 423 may include a memory device configured to access and store information associated with the computer, including information for identifying, initializing, and monitoring the operation of one or more components and subsystems. RAM 422 may include a memory device for storing data associated with one or more operations of processor 421. For example, ROM 423 may load instructions into RAM 422 for execution by processor 421.

Storage 424 may include any type of mass storage device configured to store information that processor 421 may need to perform processes consistent with the disclosed embodiments. For example, storage 424 may include one or more magnetic and/or optical disk devices, such as hard drives, CD-ROMs, DVD-ROMs, or any other type of mass media device.

Database 425 may include one or more software and/or hardware components that cooperate to store, organize, sort, filter, and/or arrange data used by the computer and/or processor 421. For example, database 425 may store digital images of a person's profile, computer-executable instructions for prediction of difficult airway management using images. It is contemplated that database 325 may store additional and/or different information than that listed above.

I/O devices 426 may include one or more components configured to communicate information with a user associated with computer. For example, I/O devices may include a console with an integrated keyboard and mouse to allow a user to maintain a database of digital images, results of the analysis of the digital images, metrics, and the like. I/O devices 426 may also include a display including a graphical user interface (GUI) for outputting information on a monitor. I/O devices 426 may also include peripheral devices such as, for example, a printer for printing information associated with the computer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 427 may include one or more components configured to transmit and receive data via a communication network, such as the Internet, a local area network, a workstation peer-to-peer network, a direct link network, a wireless network, or any other suitable communication platform. For example, interface 427 may include one or more modulators, demodulators, multiplexers, demultiplexers, network communication devices, wireless devices, antennas, modems, and any other type of device configured to enable data communication via a communication network.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

Attached hereto and fully incorporated by reference is APPENDIX A, which are select pages of a submission to the National Institute of Health (NIH) for funding to further develop the concepts described herein. APPENDIX A provides additional detailed regarding the development of the disclosed invention.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain. Unless otherwise noted, each of the below published references are fully incorporated by reference and made a part hereof:
1. Cook T M, MacDougall-Davis S R. Complications and failure of airway management. Br J Anaesth. 2012; 109 Suppl 1:i68-i85.
2. Haynes A B, Weiser T G, Berry W R, et al. A surgical safety checklist to reduce morbidity and mortality in a global population. NEnglJMed. 2009; 360(5):491-499.
3. Practice guidelines for management of the difficult airway: an updated report by the American Society of Anesthesiologists Task Force on Management of the Difficult Airway. Anesthesiology. 2003; 98(5):1269-1277.
4. Frova G, Sorbello M. Algorithms for difficult airway management: a review. Minerva Anestesiol. 2009; 75(4):201-209.
5. Norskov A K. Preoperative airway assessment—experience gained from a multicenter cluster randomised trial and the Danish Anesthesia Database. Dan Med J. 2016; 63(5).
6. Yentis S M. Predicting difficult intubation—worthwhile exercise or pointless ritual? Anaesthesia. 2002; 57(2): 105-109.
7. Shiga T, Wajima Z, Inoue T, Sakamoto A. Predicting difficult intubation in apparently normal patients: a meta-analysis of bedside screening test performance. Anesthesiology. 2005; 103 (2):429-437.
8. Norskov A K, Wetterslev J, Rosenstock C V, et al. Effects of using the simplified airway risk index vs usual airway assessment on unanticipated difficult tracheal intubation—a cluster randomized trial with 64,273 participants. Br J Anaesth. 2016; 116(5):680-689.
9. Wilson M E, John R. Problems with the Mallampati sign. Anaesthesia. 1990; 45(6):486-487.
10. Karkouti K, Rose D K, Ferris L E, Wigglesworth D F, Meisami-Fard T, Lee H. Inter-observer reliability of ten tests used for predicting difficult tracheal intubation. CanJAnaesth. 1996; 43(6):554-559.
11. Mallampati S R, Gatt S P, Gugino L D, et al. A clinical sign to predict difficult tracheal intubation: a prospective study. CanAnaesthSocJ. 1985; 32(4):429-434.
12. Frerk C M. Predicting difficult intubation. Anaesthesia. 1991; 46(12):1005-1008.
13. Naguib M, Scamman F L, O'Sullivan C, et al. Predictive performance of three multivariate difficult tracheal intubation models: a double-blind, case-controlled study. Anesth Analg. 2006; 102(3):818-824.
14. L'Hermite J, Nouvellon E, Cuvillon P, Fabbro-Peray P, Langeron O, Ripart J. The Simplified Predictive Intubation Difficulty Score: a new weighted score for difficult airway assessment. Eur J Anaesthesiol. 2009; 26(12):1003-1009.
15. Suzuki N, Isono S, Ishikawa T, Kitamura Y, Takai Y, Nishino T. Submandible angle in nonobese patients with difficult tracheal intubation. Anesthesiology. 2007; 106(5):916-923.
16. Naguib M, Malabarey T, AlSatli R A, Al D S, Samarkandi A H. Predictive models for difficult laryngoscopy and intubation. A clinical, radiologic and three-dimensional computer imaging study. CanJAnaesth. 1999; 46(8):748-759.
17. El-Boghdadly K, Onwochei D N, Millhoff B, Ahmad I. The effect of virtual endoscopy on diagnostic accuracy and airway management strategies in patients with head and neck pathology: a prospective cohort study. Can J Anaesth. 2017; 64(11): 1101-1110.
18. Connor C W, Segal S. Accurate classification of difficult intubation by computerized facial analysis. Anesth Analg. 2011; 112(1):84-93.
19. Connor C W, Segal S. The importance of subjective facial appearance on the ability of anesthesiologists to predict difficult intubation. Anesth Analg. 2014; 118(2):419-427.
20. Cormack R S, Lehane J. Difficult tracheal intubation in obstetrics. Anaesthesia. 1984; 39(11):1105-1111.
21. Huang J, Lin C X. Using AUC and Accuracy in Evaluating Learning Algorithms. IEEE Transactions on Knowledge and Data Engineering. 2005; 17(3):299-310.
22. Adnet F, Borron S W, Racine S X, et al. The intubation difficulty scale (IDS): proposal and evaluation of a new score characterizing the complexity of endotracheal intubation. Anesthesiology. 1997; 87(6):1290-1297.
23. Schroff F, Kalenichenko D, Philbin J. FaceNet: A unified embedding for face recognition and clustering. Paper presented at: 2015 IEEE Conference on Computer Vision and Pattern Recognition (CVPR), 2015; Boston, Mass.
24. https://www.nist.gov/srd/nist-special-database-18. Accessed Jul. 1, 2019.
25. https://www.kairos.com/blog/60-facial-recognition-databases. Accessed Jul. 6, 2019.
26. Goodfellow I, Pouget-Abadie J, Mirza M, et al. Generative adversarial nets. Paper presented at: Advances in neural information processing systems2014.
27. Niazi M K, Beamer G, Gurcan M N. An application of transfer learning to neutrophil cluster detection for tuberculosis: Efficient implementation with nonmetric multidimensional scaling and sampling. Submitted for publication in SPIE Medical Imaging. 2018.
28. Niazi M K K, Tavolara T E, Arole V, Hartman D J, Pantanowitz L, Gurcan M N. Identifying tumor in pancreatic neuroendocrine neoplasms from Ki67 images using transfer learning. PloS one. 2018; 13(4):e0195621.
29. Pan S J, Yang Q. A survey on transfer learning. IEEE Transactions on knowledge and data engineering. 2010; 22(10):1345-1359.
30. Torrey L, Shavlik J. Transfer learning. Handbook of Research on Machine Learning Applications and Trends: Algorithms, Methods, and Techniques. 2009; 1:242.
31. Krizhevsky A, Sutskever I, Hinton G E. Imagenet classification with deep convolutional neural networks,” in Advances in neural information processing system. Advances in neural information processing systems. 2012:1097-1105.
32. Senaras C, Niazi M K K, Arole V, et al. Segmentation of follicles from CD8-stained slides of follicular lymphoma using deep learning. Paper presented at: Medical Imaging 2019: Digital Pathology2019.
33. Srivastava N, Hinton G, Krizhevsky A, Sutskever I, Salakhutdinov R. Dropout: A Simple Way to Prevent Neural Networks from Overfitting. J Mach Learn Res. 2014; 15:1929-1958.
34. Niazi M K K, Tavolara T E, Arole V, Parwani A V, Lee C, Gurcan M N. Automated T1 bladder risk stratification based on depth of lamina propria invasion from H&E tissue biopsies: A deep learning approach SPIE Medical Imaging. 2018.
35. Niazi M K K, Tavolara T, Arole V, Parwani A, Lee C, Gurcan M. MP58-06 automated staging of T1 bladder cancer using digital pathologic h&e images: a deep learning approach. The Journal of urology. 2018; 199(4):e775.
36. Dasarathy B V. Decision fusion. Vol 1994: IEEE Computer Society Press Los Alamitos, Calif.; 1994.
37. Sun Q-S, Zeng S-G, Liu Y, Heng P-A, Xia D-S. A new method of feature fusion and its application in image recognition. Pattern Recognition. 2005; 38(12):2437-2448.
38. Liaw A, Wiener M. Classification and regression by randomForest. R news. 2002; 2(3): 18-22.
39. Belkin M, Niyogi P. Laplacian eigenmaps for dimensionality reduction and data representation. Neural computation. 2003; 15(6): 1373-1396.
40. Hanley J A, McNeil B J. A method of comparing the areas under receiver operating characteristic curves derived from the same cases. Radiology. 1983; 148(3):839-843.
41. Zou K H, O'Malley A J, Mauri L. Receiver-operating characteristic analysis for evaluating diagnostic tests and predictive models. Circulation. 2007; 115(5):654-657.
42. Hajian-Tilaki K. Sample size estimation in diagnostic test studies of biomedical informatics. J Biomed Inform. 2014; 48:193-204.
43. Apfelbaum J L, Hagberg C A, Caplan R A, et al. Practice guidelines for management of the difficult airway: an updated report by the American Society of Anesthesiologists Task Force on Management of the Difficult Airway. Anesthesiology. 2013; 118(2):251-270.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of determining difficult airways for intubation using images, comprising:
obtaining one or more digital images of a front view and/or profile view of a face of a patient;
analyzing the one or more digital images of the front view and/or the profile view of the face of the patient using a deep-learning algorithm executing on a computer to extract a plurality of facial landmarks from the one or more digital images; and
predicting, using a deep-learning model for airway prediction executing on the computer, whether the patient's airway will be difficult or easy to intubate based on the extracted plurality of facial landmarks.

2. The method of claim 1, wherein the deep-learning algorithm executing on the computer to extract the plurality of facial landmarks from the one or more digital images comprises one or more convolutional neural networks (CNNs) executing on the computer to extract the plurality of facial landmarks from the one or more digital images.

3. The method of claim 1, wherein the deep-learning algorithm executing on the computer to extract the plurality of facial landmarks from the one or more digital images comprises one or more convolutional autoencoders (CAEs) executing on the computer to extract the plurality of facial landmarks from the one or more digital images.

4. The method of claim 3, wherein the plurality of facial landmarks comprise a plurality of Farkas's anthropometric facial landmarks.

5. The method of claim 4, wherein a separate trained CAE corresponds to each of the plurality of Farkas's anthropometric facial landmarks to form an ensemble of CAEs.

6. The method of claim 5, wherein the plurality of Farkas's anthropometric facial landmarks comprise 50 Farkas's anthropometric facial landmarks.

7. The method of claim 1, wherein predicting, using the deep-learning model for airway prediction executing on the computer, whether the patient's airway will be difficult to intubate based on the extracted plurality of facial landmarks comprises using conditional generative adversarial networks (cGANs) to differentiate easy from difficult to intubate airways.

8. The method of claim 7, wherein the plurality of facial landmarks serve as a condition for the cGANs.

9. The method of claim 8, wherein two independent cGANs are trained, a first cGAN is trained to reconstruct easy to intubate airways while a second cGAN is trained to reconstruct difficult to intubate airways.

10. The method of claim 9, wherein both the first cGANs and the second cGAN reconstruct at least one of the one or more digital images from the facial landmarks and whichever cGAN reconstructs the at least one of the one or more digital images with a highest structural similarity index is used to determine whether the patient's airway will be difficult or easy to intubate.

11. A system for determining difficult airways for intubation using images, comprising:
a camera, wherein the camera obtains one or more digital images of a front view and/or profile view of a face of a patient;
a memory; and
a processor in communication with the memory, wherein the processor executes computer-executable instructions stored on the memory, the computer-executable instructions cause the processor to:
receive the one or more digital images of the front view and/or profile view of the face of a patient;
analyze the one or more digital images of the front view and/or the profile view of the face of the patient using a deep-learning algorithm to extract a plurality of facial landmarks from the one or more digital images; and
predict, using a deep-learning model for airway prediction, whether the patient's airway will be difficult or easy to intubate based on the extracted plurality of facial landmarks.

12. The system of claim 11, wherein the deep-learning algorithm to extract the plurality of facial landmarks from the one or more digital images comprises one or more convolutional neural networks (CNNs) extracting the plurality of facial landmarks from the one or more digital images.

13. The system of claim 11, wherein the deep-learning algorithm to extract the plurality of facial landmarks from the one or more digital images comprises one or more convolutional autoencoders (CAEs) extracting the plurality of facial landmarks from the one or more digital images.

14. The system of claim 13, wherein the plurality of facial landmarks comprise a plurality of Farkas's anthropometric facial landmarks.

15. The system of claim 14, wherein a separate trained CAE corresponds to each of the plurality of Farkas's anthropometric facial landmarks to form an ensemble of CAEs.

16. The system of claim 15, wherein the plurality of Farkas's anthropometric facial landmarks comprise 50 Farkas's anthropometric facial landmarks.

17. The system of claim 1, wherein predicting, using the deep-learning model for airway prediction, whether the patient's airway will be difficult to intubate based on the extracted plurality of facial landmarks comprises the computer-executable instructions causing the processor to use conditional generative adversarial networks (cGANs) to differentiate easy from difficult to intubate airways.

18. The system of claim 7, wherein the plurality of facial landmarks serve as a condition for the cGANs.

19. The system of claim 18, wherein two independent cGANs are trained, a first cGAN is trained to cause the processor to reconstruct easy to intubate airways while a second cGAN is trained to cause the processor to reconstruct difficult to intubate airways.

20. The system of claim 19, wherein both the first cGANs and the second cGAN cause the processor to reconstruct at least one of the one or more digital images from the facial landmarks and whichever cGAN reconstructs the at least one of the one or more digital images with a highest structural similarity index is used by the processor to determine whether the patient's airway will be difficult or easy to intubate.

21. The system of claim 11, wherein the camera, memory and processor comprise a smartphone.

22. A non-transitory computer-readable medium with computer-executable instructions stored thereon, said computer-executable instructions cause a processor to:
receive one or more digital images of a front view and/or profile view of a face of a patient;
analyze the one or more digital images of the front view and/or the profile view of the face of the patient using a deep-learning algorithm to extract a plurality of facial landmarks from the one or more digital images; and
predict, using a deep-learning model for airway prediction, whether the patient's airway will be difficult or easy to intubate based on the extracted plurality of facial landmarks.
